(19) United States
(12) Patent Application Publication
(10) Pub. No.: US 2008/0269589 A1
(43) Pub. Date: Oct. 30, 2008

(54) Apparatus for the Detection of Heart Activity

(75) Inventors: Jeroen Adrianus Johannes Thijs, Aachen (DE); Robert B. Elfring, Aachen (DE); Jens Muehlsteff, Aachen (DE); Olaf Such, Aachen (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 11/995,543

(22) PCT Filed: Jul. 14, 2006
(86) PCT No.: PCT/IB06/52407

§ 371 Date: Jan. 14, 2008

(30) Foreign Application Priority Data
Jul. 15, 2005 (EP) 05106544.9

(51) Int. Cl. A61B 5/05

(52) U.S. Cl. 600/407

(57) ABSTRACT

The invention relates to heart measurement and heart monitoring, in particular the measurement of mechanical heart activity, and includes a method and apparatus to using doppler radar to transmit an electromagnetic signal of a certain frequency into, and detect a reflected signal from out of, the chest of the individual, to processing the detected signal to produce an output signal representing the rate of change of the doppler signal associated with the reflected signal and to identify from the output signal a group of at least one characteristic point of the output signal, and further to calculate at least one parameter representative of heart activity, this calculation based on the at least one identified characteristic point. The apparatus provides a system for monitoring which is particularly suitable for use in the home and which does not require repeated use of impedance cardiograms which are inappropriate for use by untrained personnel.

The invention relates to a method to detect mechanical heart activity of an individual using doppler radar comprising transmitting an electromagnetic signal of a certain frequency into, and detecting a reflected signal from out of, the chest of the individual, and processing the detected signal to produce an output signal representing the rate of change of the doppler signal associated with the reflected signal, the rate of change with respect to time.

The use of frequency modulated doppler radar to measure heart rate is known. U.S. Pat. No. 4,958,638, for example, describes a vital signs monitor utilizing a frequency modulated doppler radar beam which when trained on the surface of the chest from a distance provides a measurement of heart rate. The frequencies of 3 and 10 GHz used for the vital signs monitor are reported as having minimal penetration into the body.

‘Less Contact: Heart-rate detection without even touching the user’ by Florian Michahelles, Ramon Wicki and Bernt Schiele, Eighth International Symposium on Wearable Computers, ISWC 2004, Volume: 1, pp. 4-7, 31 Oct.-3 Nov., 2004, describes a system to measure heart-rate using micro impulse radar pulses. The detected signal is filtered and the distances between all local maxima calculated and analyzed for regularly occurring patterns. All maxima occurring within a certain distance are presumed to stem from the heart beat and are used to derive the heart rate.

U.S. Pat. No. 4,967,751 describes a system for measuring breathing rate using the transmission of a continuous frequency electromagnetic wave through the upper body of a human being, the detection of the doppler shifted signal on the other side of the upper body, the frequency modulation of this detected signal and its retransmission back through the upper body and eventual detection at the original transducer. The signal contains cyclical information about the breathing rate of the person. Further, the frequency modulation of the doppler shifted signal allows the required signal to be identified with respect to any other stray signals detected by the original transducer. These stray signals may originate from, for example, back-scattering of the original signal by organs in the body, for example the heart or lungs. U.S. Pat. No. 4,967,751 discloses that the movement of these organs introduces a doppler-frequency component into the back-scattered signal and explains that this may originate from the breathing rate, the beating rate of the heart and the movement of the heart valves.

U.S. Pat. No. 3,483,860 describes a method for monitoring heart movement comprising transmitting a radio frequency signal into the body and detecting and processing the reflected signal to produce an output signal. The output signal is further differentiated to provide an indication of rate of ejection of blood from the heart.

It is an object of the invention to provide an improved measurement of mechanical heart activity.

This is achieved according to the invention whereby the method further comprises the steps of identifying from the output signal a group of at least one characteristic point of the output signal, and further calculating at least one parameter representative of heart activity, the calculation based on the at least one identified characteristic point.

The method includes transmitting an electromagnetic signal into the chest of an individual which is then reflected back from any internal organs in its path. The electromagnetic signal becomes doppler shifted in the event that a reflecting organ is moving relative to the transducer. This doppler shifted signal is detected by the transducer and when visually displayed shows a cyclical behavior representative of heart activity. However, if this signal is processed by a processor to produce the rate of change of the signal with respect to time it is found that this outputted signal contains information which allows information about mechanical heart activity to be extracted from the further signal.

Specifically, the further signal contains cyclically occurring features and surprisingly, when the further signal is compared to a trace from an impedance cardiogram it can be seen that equivalents of characteristic points found on the trace of the impedance cardiogram can be identified on the further signal, allowing parameters such as pre-ejection period and left ventricular ejection time, which are normally calculated using the impedance cardiogram, to be calculated using the outputted signal. Therefore information representing mechanical activity of the heart can be extracted from the outputted signal and parameters can be calculated which provide a measure of mechanical heart activity. The method requires no impedance cardiogram to be performed and yet still allows the same parameters to be calculated. Equipment to perform the method is easier to use, requiring simple placement of the transducer against the chest, and is therefore more suitable for repeated measurement of heart activity and is correspondingly more suited to repeated measurements, for example in patient monitoring.

The invention also relates to a system to detect mechanical heart activity of an individual using doppler radar comprising a transducer, to transmit electromagnetic signals of a certain frequency into, and detect reflected signals from out of, the chest of the individual, a first computer processor, coupled to the transducer, to process the detected signal to produce an output signal representing the rate of change of the doppler signal associated with the reflected signal, the rate of change with respect to time, a second computer processor arranged to identify from the output signal a group of at least one characteristic point of the output signal, and a third computer processor arranged to calculate at least one parameter representative of heart activity, the calculation based on the at least one identified characteristic point. This system has the advantage that it allows the method of the invention to be performed over multiple devices and thereby provide maximum flexibility in assessing the mechanical activity of the heart of an individual. The computer processors can be situated within the same computer or be geographically separate from each other. In the latter case, transmission of information between the processors can be accomplished by any known wireless means, or by modem connection, or by known computer network technology.

The invention also relates to a wearable apparatus to detect mechanical heart activity of an individual using doppler radar, comprising a transducer to transmit electromagnetic signals of a certain frequency into the chest of the individual, and to detect reflected signals from out of the chest, and to transmit a signal representative of the detected signal to be received by a processing system, which system is arranged to use the received signal to calculate an output signal representing the rate of change of the doppler signal associated with the reflected signal, the rate of change with respect to time, to identify from the output signal a group of at least one characteristic point of the output signal, and to calculate at least one parameter representative of heart activity, the calculation based on the at least one identified characteristic point.

This apparatus has the advantage that it can be worn by an individual while they move around and can therefore acquire signals demonstrating mechanical heart activity while the individual is ambulatory. It has the further advantage that the wearable apparatus need only comprise a suitable transducer for the production of electromagnetic signals and need not comprise the processor, which may itself be remote from the wearable apparatus, thereby saving space and weight in the wearable apparatus. Thus the wearable apparatus has the advantage that it provides output signals to a remote processor which calculates the rate of change of the originally detected signal with respect to time, identifies the characteristic points and calculates parameters. The remote processor may be physically located in the same room as the individual, or may even be located in another room in the same house.

The wearable apparatus can be worn by the individual on a strap or a harness or using other carrying means. Because the electromagnetic signals can penetrate through cloth and other wearable materials the apparatus can also be carried in a pocket constructed on the clothing of the individual and arranged to be situated in a position where an optimal signal is detected by the transducer.

The invention also relates to a processing system, for receiving the signal transmitted from a wearable apparatus to detect mechanical heart activity of an individual using doppler radar, the system arranged to receive a signal representative of a reflected electromagnetic signal detected from out of the chest of an individual, and further arranged to calculate an output signal representing the rate of change of the doppler signal associated with the reflected signal, the rate of change with respect to time, identify from the output signal a group of at least one characteristic point of the output signal, and calculate at least one parameter representative of heart activity, the calculation based on the at least one identified characteristic point.

This apparatus has the advantage that it processes the signals from a portable apparatus arranged to detect doppler radar signals from within the chest of an individual and processes them to produce signals representative of mechanical heart activity according to the method of the invention.

Thus the wearable apparatus in combination with the remote processor together offer a solutions which solves the problem of how to arrange for ambulatory monitoring of mechanical heart activity of the individual.

The invention also relates to a system for the ambulatory detection of mechanical heart activity of an individual using doppler radar, comprising a transducer to transmit an electromagnetic signal of a certain frequency, the transducer positioned so that the doppler radar signal is emitted into the chest of the individual, the transducer capable of detecting the reflected signal from out of the chest, and further arranged to transmit a signal representative of the detected signal, a first remote computer processor arranged to receive the signal representative of the detected signal, and arranged to:

process the detected signal to produce an output signal representing the rate of change of the doppler signal associated with the reflected signal, the rate of change with respect to time, a second remote computer processor arranged to identify from the output signal a group of at least one characteristic point of the output signal, and a third remote computer processor arranged to calculate at least one parameter representative of heart activity, the calculation based on the at least one identified characteristic point.

The system has the advantage that it allows the ambulatory monitoring of mechanical heart activity using a wearable transducer which emits electromagnetic signals and detects doppler shifted reflections of those signals, passes those signals to a series of remote processors, and processes those signals to produce a signal representative of mechanical heart activity. The remote processors, for example, may be in the same room as the individual and may even be in the same computer, but could be in another room in the same building or separated from each other geographically.

This system also has the further advantage that it can be used to provide monitoring of mechanical heart activity using a world wide web service. In this case, the individual who is monitored wears the transducer in a housing, arranged in some way on his or her person, as above, so that a suitable signal is detected which has been reflected from the heart, and the processor which calculates the rate of change of the detected signal is contactable via the world wide web. In this case the skilled person can arrange for the signal from the wearable apparatus to be transmitted to an intermediate processor, a computer with a connection to the world wide web, say, which is arranged to transmit the signal representative of the detected signal through the world wide web to the remote processor. Alternatively, the wearable apparatus can be equipped with suitable processing to allow for the direct transmission of the signal representative of the detected signal into the world wide web to the remote processor.

Thus the system solves the problem of how to provide monitoring of mechanical heart activity from a location remote from the location of the individual being monitored.

The apparatus of the invention is particularly advantageously arranged when it emits continuous wave electromagnetic waves, although as a feature this is not necessary. The apparatus of the invention achieves the desired result if the emitted and reflected signal is of such a duration that it is able to encode information from at least a single heart beat. This can definitely be achieved if the electromagnetic signals are emitted in the form of a continuous beam. However, pulsed electromagnetic signals can also be used if each single pulse is long enough to encode the information from a single heart beat, or, for example, if the time interval between pulses is very short in comparison with the time it takes the heart to beat once. In the later case, each pulse encodes some fraction of the information available in each heart beat about the heart activity. In the case where a train of very short pulses with a very short time interval are used the information encoded in the doppler shifted reflected signals represents a sampling of information from the heart.

The apparatus of the invention can be used with a transducer arranged to produce electromagnetic signals of frequency in a range of between 400 MHz and 5 GHz. This range produces reflected signals from the heart. However, the apparatus works in a particularly advantageous manner when the frequency is in a range of between 800 MHz and 4 GHz.

The apparatus is operated advantageously when it emits electromagnetic signals which are of a single frequency, within the limits of conventional operation of electromagnetic antenna, as will be appreciated by the person skilled in the art.

The invention is further elucidated and embodiments of the invention are explained using the following figures.

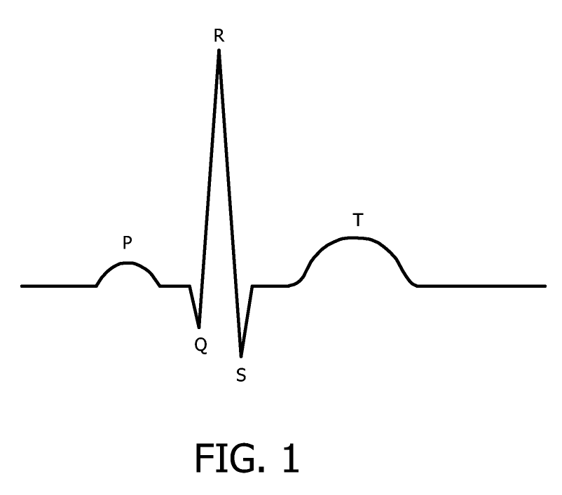

FIG. 1 shows a typical trace from an ECG measurement of the heart.

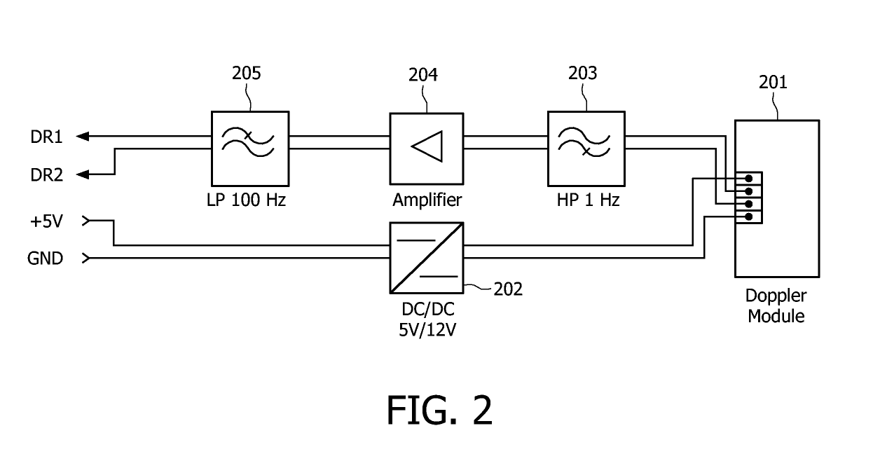

FIG. 2 shows a block diagram of the apparatus of the invention.

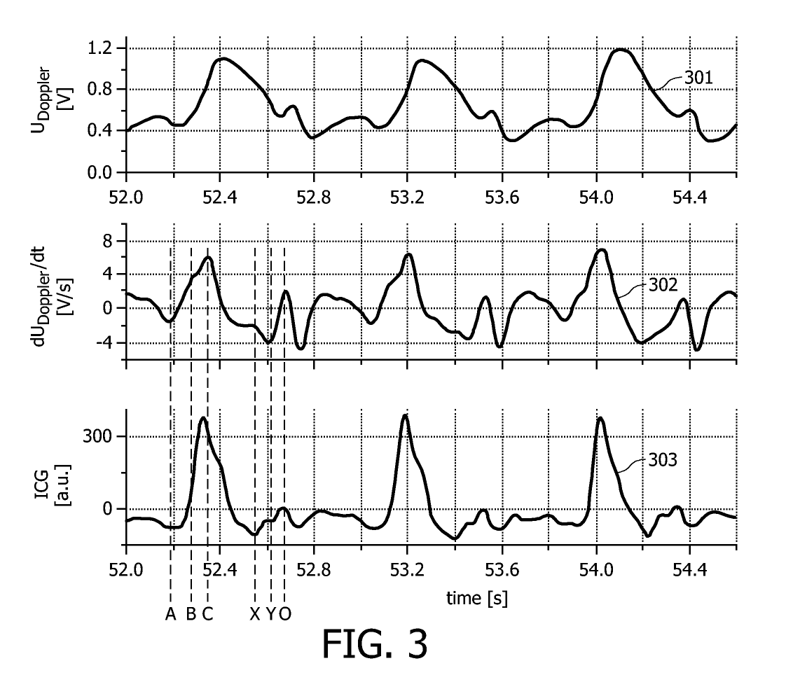

FIG. 3 shows the output of the processor which processes the signal detected by the transducer.

As is commonly known, the heart is the organ which pumps blood around the body. It is subdivided into 4 chambers, consisting of 2 atria, which receive blood entering the heart, with deoxygenated blood returning from the body entering into the right atrium and oxygenated blood from the lungs entering into the left atrium, and 2 larger ventricles which are responsible for pumping blood out of the heart. The right ventricle pumps deoxygenated blood received from the right atrium out of the heart and to the lungs, where it is oxygenated. The left ventricle, the largest chamber in the heart, is responsible for pumping oxygenated blood received from the left atrium out into the rest of the body. As is also known, measurements from electrocardiography, ECG, show that the heart pumps in a cyclical fashion and ECG measurements allow identification of certain phases common to the electrical sequence of the heart. FIG. 1 shows a typical output trace from an ECG measurement. The characteristic spikes shown in a typical trace are labeled P, Q, R, S and T, as indicated. It is known that the P spike, or wave, is representative of the depolarization, or excitation, of the atria. The QRS spikes, known commonly as the QRS-complex, are representative of the excitation of the ventricles. The QRS-complex masks any signal from the repolarisation of the atria. The T spike, or T wave, is representative of the repolarisation of the ventricles.

Transducers for the detection of doppler shifted signals are commercially available, and are often used for the purposes of detection of movement using the far field of the beam, for example in Radar measurements of traffic speed. It is now found, according to the invention that such transducers can also be used for near field measurements and are surprisingly suitable for detecting mechanical heart activity via the detection of doppler shifted signals from the heart.

Generally in such doppler transducers, as is known in the art, an antenna emits an electromagnetic wave which, when it is reflected from the surfaces of an object moving with a component of velocity non-transverse to the impinging electromagnetic wave, produces a shift in the frequency of the electromagnetic wave reflected back to the antenna. This shift in frequency is called the doppler shift. This doppler shifted reflected wave is detected by an antenna in the transducer, which may or may not be the same antenna as the emitting antenna. The relative speed of movement of the reflecting object is encoded in the frequency shift of the detected reflected wave and this value can be extracted using known techniques.

A transducer advantageously used in the apparatus of the invention contains a 2.45 GHz oscillator operating in continuous mode. It is known that electromagnetic radiation is strongly absorbed in human tissue at around the frequencies of 2 to 10 GHz, but it is found, according to this highly advantageous embodiment of the invention, that the radiation produced from an antenna operating at 2.45 GHz, although absorbed and scattered to some extent by layers of tissue, produces a detectable signal.

A particularly advantageous embodiment utilizes a commercially available Microwave Motion Sensor KMY 24 unit made by Micro Systems Engineering GmbH. It contains a 2.45 GHz oscillator and receiver in the same housing and works in continuous wave mode. The dimensions of the beam are, amongst other things, dependent on the dimensions of the antenna and in this case the unit contains an optimized patch antenna with minimized dimensions and a width of 3.5 cm, producing a beam with a near field radius of 2 cm. This provides a workable compromise between too large an antenna, which would produce a wide beam easily contaminatable by reflections from other structures, and too small an antenna, which would produce a narrow beam which is difficult to position satisfactorily. In practice, a beam with a width in the range of 1 cm to 2.5 cm is advantageous because it provides a workable compromise between the two extremes described above. A beam with a width in the range of 1.5 cm to 3 cm is particularly advantageous for application of the apparatus to large adults or adults with an enlarged heart. A beam with a width in the range of 0.5 cm to 1.75 cm is advantageous for application of the apparatus to small children.

The commercially available unit is utilized in the following way. FIG. 2 shows a block diagram of the apparatus. The doppler transducer 201 is powered by a voltage supply 202. The output of the doppler transducer 201 is processed through a high pass filter 203, a preamplifier 204 and a low pass filter 205. It was found experimentally that the high pass filter 203 should comprise a capacitance of 100 nF and a resistor of 1 MΩ, as this enabled a faster decay of the signal while removing the DC part of the signal from the doppler module. The time constant τ of 0.1 s produces a cut-off frequency of 1.59 Hz. Although the signal being detected is reflected from the heart which beats with a frequency of the order of 1 Hz, the attenuation of this first order high pass filter is low enough not to destroy the signal. The gain of the preamplifier 204 can be set in a range of 1 to 1000 but it was found that a particularly advantageous gain was 500. To enable sampling, an 8^{th} order low pass filter was realized with a cutoff frequency of 100 Hz using operational amplifiers.

FIG. 1 also shows two output signals, DR1 and DR2, from the doppler transducer. As is known in the art, some commercially available transducers contain two mixer diodes to provide additional information about the direction of movement of the reflecting object. However, two signals are not necessary for the apparatus to work. If such a transducer is used to construct the apparatus the reflected signal from either mixer diode can be used for the calculation of rate of change.

It was found that the whole assembly is sensitive enough to process signals that are reflected by the heart.

Experimental results show that the positioning of the transducer relative to the heart is important in detecting a useful signal. The electromagnetic signals must be reflected from the heart itself in order for mechanical heart information to be encoded in the reflected signals. However, it is found experimentally that individual variation between subjects alters the correct position or positions of the transducer in respect of optimal signal detection for each individual. However, if both the detected and output signals are visually displayed on a display screen it is possible to see if the transducer is correctly placed. If the transducer is placed in such a way that the heart is not in the emitted beam of signals, or is not reflecting the emitted signals back to the receiver, little or no cyclical activity will be seen in the reflected beam. If the transducer is well positioned a cyclical signal will be seen. A certain amount of experimentation is required in the correct positioning of the transducer on the surface of the chest of the individual before a suitable signal and therefore the correct position identified. It has been found that arranging the sensor so that the emitted beam impinges on a plane structure predominantly parallel to the plane of the transducer, for example a section of heart wall muscle, is highly advantageous in receiving an adequate reflected signal.

The transducer can be incorporated in a suitable housing which is advantageously dimensioned so that it can be arranged flat against the chest, for example the sternum of the individual. Suitable dimensions are between 3 and 6 cm wide and between 4 and 7 cm long. These sizes allow for the hardware to be contained in the housing while maintaining the housing at a size which can be used effectively on an individual.

The technical steps to be performed in the processing of the recorded data to provide an output signal containing the rate of change of the data with respect to time can be undertaken by a person skilled in the art using known data processing techniques. For example, it can be achieved using the Matlab computer language.

Similarly, the method used to extract a signal representative of the rate of change of the signal with respect to time will be known to the person skilled in the art. For example, the signal can be sampled and the rate of change of each sample over the length of the sample extracted. However, the output signal can also be calculated by inverse transforming the detected signal to derive the mathematical function of the signal and mathematically deriving the function to produce the first order derivative.

FIG. 3 shows the output of the processor which processes the signal detected by the transducer. The first trace 301 is the detected signal. The second trace 302 is the rate of change of the detected signal with respect to time. The third trace 303 is an example of a trace from an impedance cardiogram. It can be seen from FIG. 3 that the characteristic points of the impedance cardiogram 303 can be similarly identified on the trace representing the rate of change of the detected signal. Specifically these characteristic points, known to the skilled person, are:
A: representing the contraction of the atrium
B: representing the opening of the aortic valve and the beginning of the systolic ejection phase
C: representing maximum systolic flow
X: representing the closing of the aortic valve and the end of the ejection phase
Y: representing the closing of the pulmonal valve
O: representing the opening of the mitral valve

In other words, points equivalent to known characteristic points identifiable from an impedance trace are now also identifiable from a signal which is the rate of change of a detected doppler signal reflected from the heart of an individual.

The characteristic points can be identified using known techniques of signal processing and is a matter of design for the person skilled in the art. For example, the characteristic points can be identified from analysis of the morphology of the rate of change trace 302.

It was further found experimentally that characteristic point A, which is normally not very clearly identifiable in an impedance cardiagram, is more easily distinguishable using the apparatus of the invention and the technical features detailed in claim 1.

Using these characteristic points several parameters can be calculated, as is commonly known in the art, but see for example user manual for the pc-software of the publically available ‘Niccomo’ hemodynamic monitor, supplied originally by Medis GmbH, now owned by CardioDynamics, Section D, ‘Description of the calculated parameters’, pages 55-64, detailing commonly known clinically relevant parameters and details of their calculation using the known characteristic points. These parameters include pre-ejection period, left ventricular ejection time, systolic time ratio and ejection time ratio. The parameter of left ventricular ejection time is sometimes referred to in the art as left ventricular ejection phase. The calculation of these parameters proceeds along the same lines as for their calculation using the prior art method of impedance cardiography and is therefore not the subject of this invention. However, as can be seen from the Niccomo user manual, calculation of these parameters in the prior art requires characteristic points derived from an impedance cardiogram. The invention provides a measure of mechanical heart activity which provides improved information concerning heart movement.

The computer processing arranged to calculate the doppler signal, calculate the rate of change of the doppler signal, identify the characteristic points and then calculate the parameters from the characteristic points can be situated in various items of equipment. Although the transducer itself will of necessity be positioned, when in use, in such a way that a doppler signal is produced which encodes information about the heart, the processing that occurs after the transducer has received the initial signal need not be physically coupled to the transducer but may be arranged to received the output of the transducer wirelessly using any known wireless means. Similarly, the stages of processing may be separated and undertaken in processing units which are situated physically apart from each other but arranged to relay or transmit their results to each other using any known method including, for example, wireless transmission, transmission down a telephone line or, say, along a fixed, physical connection such as a wire.

As an example of how the invention may be worked, the individual whose heart activity is to be measured is provided with a wearable doppler transducer fitted into a comfortable harness and coupled to a transmitter arranged to transmit the detected signal to a first remote processor which performs the actions of processing the signal to produce a doppler signal, calculating the rate of change of this doppler signal, identifying the characteristic points and using these to calculate any required parameters. In the case when the resulting first processor is in the same location as the individual, for example their home, residence or hospital ward, this first processor can be arranged to further transmit the resulting parameters, along with the doppler trace and/or a trace of the rate of change of the doppler signal as appropriate, to a remote second processor situated in a computer workstation. The results can be accessed at this workstation by a doctor or other medical professional for the purposes of monitoring the health of the individual.

Alternatively, the first processor could be arranged to calculate only the doppler signal and communicate this to the second processor which can itself be arranged to perform all further analysis.

Alternatively, the first processor could be arranged to calculate only the doppler signal and calculate the rate of change of this doppler signal and then communicate this to the second processor which can itself be arranged to perform all further analysis. In this sense the step of processing the detected signal to produce an output signal representing the rate of change of the doppler signal associated with the reflected signal, can as an example be performed by first processing the detected signal to produce a doppler signal and then processing the doppler signal to produce an output signal representing the rate of change of the doppler signal with respect to time. In fact calculation of the doppler signal itself is not strictly necessary as an intermediate step and other methods of calculating this rate of change of the doppler signal may be performed by the person skilled in the art as a matter of design, once he understands that it is the rate of change of the doppler signal information which allows identification of the characteristic points.

Alternatively, the first processor could be arranged to calculate the rate of change of the doppler signal and identify the characteristic points and then communicate these to the second processor which is arranged to perform the further analysis.

Alternatively, the first processor could be arranged to calculate the rate of change of the doppler signal, identify the characteristic points and then calculate the parameters, communicating any combination of these to a further processor or workstation where the results can be examined by a doctor or other medical professional.

In an alternative embodiment to the wireless transfer of information between the transducer and processor, the transducer may store the information contained in the detected signal for transfer to a processor via a docking station or other fixed connection after the measurement session is complete. This removes the need for wireless capability and thereby reduces the possibility of signal interference in an environment with a inherently large electromagnetic signal load.

Alternatively, the transducer may remain connected to the processor via a fixed connection, such as a lead, during the measurement session. This also reduces the possibility of interference while allowing interim results to be calculated during the measurement session. This provides advantages in the case whereby the individual experiences a sudden increase in symptoms and it becomes desirable to communicate information regarding the mechanical activity of the heart urgently to a medical professional.

In an exemplary embodiment, the transducer, situated in a comfortable harness is used by the individual once a day for a short period of time, say 5 mins, to take readings of the activity of the heart. The resultant data, either as raw data or as identified characteristic points or as calculated parameters is transmitted to a geographically remote location where it is analyzed by a doctor or other medical professional for monitoring of the individual over time.

In this case the correct position of the transducer on the chest of the individual can be initially identified using an initially performed impedance cardiogram. Thereafter, the individual simply places the transducer in the correctly identified position at regular intervals, say once a day, and operates it himself to provide parameters which provide information concerning the mechanical activity of his heart. The resulting information is advantageously used when communicated to a doctor or health monitoring service, but could also be transmitted directly to a processor which is part of a computer aided detection system designed to automatically monitor the individual's health and alert him or a doctor or a health monitoring service in the event that the calculated parameters indicate a deterioration in the individual's condition.

It can be seen in the light of the information above that the invention provides a system for monitoring which is particularly suitable for use in the home and which does not require repeated use of impedance cardiograms which are inappropriate for use by untrained personnel.

1. A method to detect mechanical heart activity of an individual using doppler radar comprising:
transmitting an electromagnetic signal of a certain frequency into, and detecting a reflected signal from out of, the chest of the individual,
transmitting a signal representative of the detected signal to a processing system,
processing, by processing system the detected signal to produce an output signal representing the rate of change of the doppler signal associated with the reflected signal, the rate of change with respect to time, wherein the method further comprises the steps of:
identifying, by the processing system from the output signal a group of at least one characteristic point of the output signal,
and further calculating, by the processing system at least one parameter representative of heart activity, the calculation based on the at least one identified characteristic point,
wherein the signal representative of the detected signal is transmitted wirelessly to a computer with a connection to the world wide web and from said computer through the world wide web to the processing system.

2. (canceled)

3. A wearable apparatus to detect mechanical heart activity of an individual using doppler radar, comprising:
a transducer to transmit electromagnetic signals of a certain frequency into the chest of the individual, and to detect reflected signals from out of the chest,
and to transmit a signal representative of the detected signal to a processing system, which system is arranged to use the received signal to calculate an output signal representing the rate of change of the doppler signal associated with the reflected signal, the rate of change with respect to time,
identify from the output signal a group of at least one characteristic point of the output signal,
and calculate at least one parameter representative of heart activity, the calculation based on the at least one identified characteristic point,
wherein the apparatus comprises means for transmitting the signal representative of the detected signal wirelessly to a computer with a connection to the world wide web, wherein said computer is adapted to further transmit the signal representative of the detected signal through the world wide web to the processing system.

4. A processing system, for receiving a signal transmitted from a wearable apparatus to detect mechanical heart activity of an individual using doppler radar, the system arranged to receive a signal representative of a reflected electromagnetic signal detected from out of the chest of an individual, and further arranged to:
calculate an output signal representing the rate of change of the doppler signal associated with the reflected signal, the rate of change with respect to time,
identify from the output signal a group of at least one characteristic point of the output signal,
and calculate at least one parameter representative of heart activity, the calculation based on the at least one identified characteristic point,
wherein the processing system is adapted to receive the signal representative of the detected signal through the world wide web from a computer with a connection to the world wide web, wherein said computer is adapted to receive the signal representative of the detected signal wirelessly from the wearable apparatus.

5. (canceled)

6. The apparatus as claimed in claim 3, wherein the rate of change of the detected signal with respect to time is calculated as the first order derivative of the detected signal with respect to time.

7. The apparatus as claimed in claim 3, wherein the transducer is arranged to emit continuous wave electromagnetic signals.

8. The apparatus as claimed in claim 3, wherein the transducer emits continuous wave electromagnetic signals at a frequency in a range between 400 MHz and 5 GHz.

9. The apparatus as claimed in claim 8, wherein the transducer emits continuous wave electromagnetic signals at a frequency in a range between 800 MHz and 4 GHz.

10. The apparatus as claimed in claim 9, wherein the transducer emits continuous wave electromagnetic signals at a frequency of 2.45 GHz.

11. The apparatus as claimed in claim 3, wherein it further comprises a display screen for the display of the output signal.

12. The apparatus as claimed in claim 3, wherein the parameters representative of heart activity include at least one of pre-ejection period, left ventricular ejection time, systolic time ratio and ejection time ratio.

13. The apparatus as claimed in claim 3, wherein it is further arranged to output the value of at least one calculated parameter representative of heart activity.

13. The method of claim 1, wherein the at least one parameter representative of heart activity is transmitted to a computer aided detection system designed to automatically monitor the individual's health and output and alert in the event that the parameter indicates a deterioration in the individual's condition.
